(19) United States
(12) Patent Application Publication
(10) Pub. No.: US 2009/0068743 A1
(43) Pub. Date: Mar. 12, 2009

(54) CATIONIC ALPHA-AMINO ACID-CONTAINING BIODEGRADABLE POLYMER GENE TRANSFER COMPOSITIONS

(75) Inventors: William G. Turnell, San Diego, CA (US); Gina Ann Cruz-Aranda, Bonita, CA (US); Mark MinZhi Wu, San Diego, CA (US); Ronald Lee Chantung, San Diego, CA (US); Zaza D. Gomurashvili, La Jolla, CA (US); Kristin M. DeFife, San Diego, CA (US)

Correspondence Address: DLA PIPER LLP (US), 4365 EXECUTIVE DRIVE, SUITE 1100, SAN DIEGO, CA, 92121-2133, US

(73) Assignee: MediVas, LLC, San Diego, CA (US)

(21) Appl. No.: 12/197,068

(22) Filed: Aug. 22, 2008

Related U.S. Application Data
(60) Provisional application No. 60/957,664, filed on Aug. 23, 2007.

(51) Int. Cl. C12N 15/87; C12N 15/11

(52) U.S. Cl. 435/455; 536/23.1

(57) ABSTRACT

The invention provides gene transfer compositions using as the gene carrier a biodegradable polymer that contains one or more cationic alpha amino acids, such as arginine or agmatine. The compositions form a tight soluble complex with a poly nucleic acid suitable for transfecting target cells to effect translation of the cargo poly nucleic acid by the target cell. Thus, such compounds are useful both in vitro and in vivo.

### RELATED APPLICATIONS

This application claims priority under 35 U.S.C. §119(e) of U.S. Provisional application Ser. No. 60/957,664 filed Aug. 23, 2007 which is hereby incorporated by reference in its entirety.

### BACKGROUND OF THE INVENTION

Gene therapy can be defined as the treatment of disease by the transfer of genetic material into specific cells of a subject. The concept of human gene therapy was first articulated in the early 1970s. Advances in molecular biology in the late 1970s and throughout the 1980s led to the first treatment of patients with gene-transfer techniques under approved FDA protocols in 1990. With optimistic results from these studies, gene therapy was expected to rapidly become commonplace for the treatment and cure of many human ailments. However, considering that 1131 gene-therapy clinical trials have been approved worldwide since 1989, the small number of successes is disappointing.

The genetic constructs used in gene therapy consist of three components: a gene that encodes a specific therapeutic protein; a plasmid-based gene expression system that controls the functioning of the gene within a target cell; and a gene transfer system that controls the delivery of the gene expression plasmids to specific locations within the body. A key limitation to development of human gene therapy remains the lack of safe, efficient and controllable methods for gene transfer.

The use of viral vectors for human clinical use has historically encountered limitations, which may range from limited payload capacity and general production issues to immune and toxic reactions, as well as the potential for undesirable viral recombination. Polymers and lipids are the most common non-viral synthetic transfer vectors and have been developed in an effort to avoid the possibility of such limitations. Therefore, non-viral systems, especially synthetic DNA delivery systems, have become increasingly desirable in both research laboratories and clinical settings.

However, research in the field of non-viral gene transfer is in its infancy compared to research of viral-based gene transfer systems. In recent years many groups have used protein-transduction domains (PDT) to enhance intracellular delivery of cargoes; a well studied example being the arginine-rich segment of the transactivator of transcription for HIV-1, TAT. In related studies, it was found that the TAT sequence could be displaced with a monomer of arginine, showing that the guanidinium residues of arginine are essential to the ability of TAT to transfer a heterologous gene into a target cell. Since this discovery, many groups have prepared chemical conjugates of guanidine-rich PTDs with drugs, oligonucleotides, proteins, nanoparticles, and liposomes and successfully delivered them into a broad variety of cell types. In addition, molecular arginine has been suggested for pharmacological use as an anticoagulant and arginine conjugated to the natural polymer chitosan has also been reported (W G Liu et al. J. Mat. Sci.: Materials in Medicine (2004) 15).

Among the common cationic polymers that have been evaluated as a non-viral gene transfer agent, the best known are poly-L-lysine (PLL) and polyethylenimine (PEI). Other synthetic and natural polycations that have been developed as non-viral vectors include polyamidoamine dendrimers (Tomalia, D. A., et al. Angewandte Chemie-International Edition in English (1990) 29(2)” 138-175) and modified chitosan (Erbacher, P., et al. Pharmaceutical Research (1998) 15(9):1332-1339).

Polymers that have been specifically designed to improve gene transfer efficiency include imidazole-containing polymers with proton-sponge effect, membrane-disruptive peptides and polymers, such as polyethylacrylic acid (PEAA) and polypropylacrylic acid (PPAA); cyclodextrin-containing polymers and degradable polycations, such as poly[alpha-(4-aminobutyl)-L-glycolic acid] (PAGA) and poly(amino acid); and polycations linked to a nonionic water-soluble polymer, such as polyethylene oxide (PEO). In most cases, these polymers were designed to address a specific intracellular barrier, such as stability, biocompatibility or endosomal escape. The results have been mixed, with some polymers performing as well as, or even slightly better than, the best off-the-shelf polymers. However, none approach the efficiency of viruses as a gene transfer vector.

During the past decade, biodegradable, bioresorbable polymers for biomedical uses have garnered growing interest. Recently described, aliphatic PEAs based on α-amino acids, aliphatic diols, and fatty dicarboxylic acids have been found to be good candidates for biomedical uses because of their biocompatibility, low toxicity, and biodegradability (K. DeFife et al. Transcatheter Cardiovascular Therapeutics—TCT 2004 Conference. Poster presentation. Washington, D.C. 2004; G. Tsitlanadze, et al. J. Biomater. Sci. Polymer Edn. (2004). 15:1-24).

The highly versatile Active Polycondensation (APC) method, which is mainly carried out in solution at mild temperatures, allows synthesis of regular, linear, polyfunctional PEAs, poly(ester-urethanes) (PEURs) and poly(ester ureas) (PEUs) with high molecular weights. Due to the synthetic versatility of APC, a wide range of material properties can be achieved in these polymers by varying the three components—α-amino-acids, diols and dicarboxylic acids—used as building blocks to fabricate the macromolecular backbone (R. Katsarava, et al. J Polym. Sci. Part A: Polym. Chem (1999) 37:391-407). Recently it has been discovered that cationic PEAs that incorporate arginine into the polymer backbone can be used as a non-viral gene transfer agent (U.S. provisional Application 60/961,876, filed Jul. 24, 2007).

The above studies have shown that there are three major barriers to efficient DNA delivery: low uptake across the cell plasma membrane; inadequate release and instability of released DNA molecules, and difficulty of nuclear targeting. Thus, despite the above described advances in the art, there is a need for new and better non-viral gene transfer systems.

### SUMMARY OF THE INVENTION

In one embodiment the invention provides a biodegradable gene transfer composition comprising at least one poly nucleic acid condensed into a soluble complex with a cationic polymer comprising at least one of the following:
a PEA polymer having a chemical formula described by general structural formula (I),

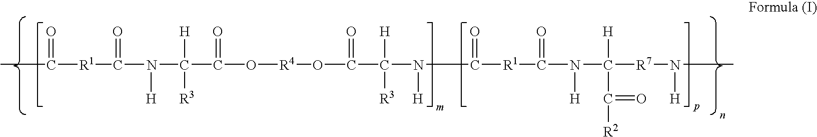

wherein n ranges from about 15 to about 150, m ranges about 0.1 to 0.9; p ranges from about 0.9 to 0.1;
R¹ is independently selected from the group consisting of (C₂-C₂₀) alkylene, (C₂-C₂₀) alkenylene, α,ω-bis(4-carboxyphenoxy)-(C₁-C₈) alkane, and α,ω-alkylene dicarboxylates of structural formula (II) and combinations thereof; wherein R⁵ in Formula (II) is independently selected from (C₂-C₁₂) alkylene, and (C₂-C₁₂) alkenylene, and R⁶ in Formula (II) is independently selected from the group consisting of (C₂-C₁₂) alkylene, (C₂-C₁₂) alkenylene, and (C₂-C₈) alkyloxy (C₂-C₂₀) alkylene,

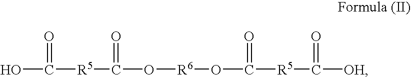

R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and

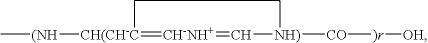

(polyhistidine), wherein r ranges from about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (III), and combinations thereof; and

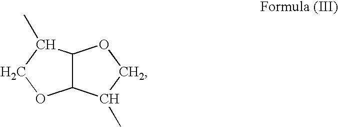

R⁷ is independently (C₂-C₂₀) alkyl or (C₂-C₂₀) alkenyl;

or a PEUR polymer having a chemical structure described by general structural formula (IV)

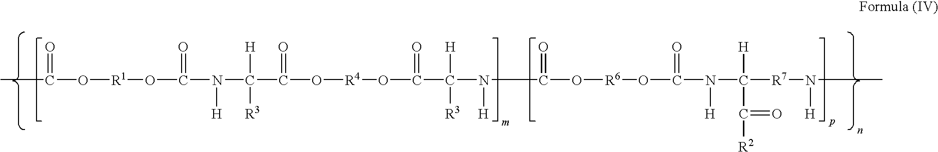

wherein n ranges from about 15 to about 150, m ranges from about 0.1 to 0.9; p ranges from about 0.9 to 0.1;
R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and

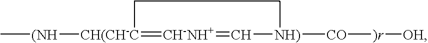

(polyhistidine), wherein r ranges from about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ and R⁶ are each independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof; and
R⁷ is independently (C₂-C₂₀) alkyl or (C₂-C₂₀) alkenyl;

or a PEU polymer having a chemical formula described by general structural formula (V):

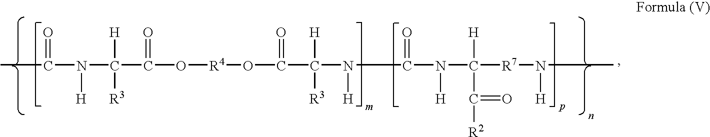

wherein n ranges from about 15 to about 150, m ranges about 0.1 to 0.9; p ranges from about 0.9 to 0.1;
R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and

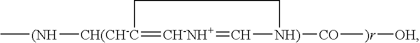

(polyhistidine), wherein r ranges from about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II); and
R⁷ is independently (C₂-C₂₀) alkyl or (C₂-C₂₀) alkenyl.

In another embodiment, the invention provides methods for transfecting a target cell by incubating the target cell in solution with the invention gene transfer composition so as to transfect the target cell with the poly nucleic acid condensed therein.

### A BRIEF DESCRIPTION OF THE FIGURES

FIG. 1 is a graph showing percent viability of FL83B cells in the presence of PEA-Arg(OMe).HCl, PEA-Arg(OMe).AA, and PEA-Agmatine.AA at various polymer concentrations. Of the three polymers, PEA-Arg(OMe) conjugates were the least toxic to FL83B cells.

FIG. 2 is a graph showing percent viability of FL83B cells in the presence of various concentrations of polymers PEA-NTA-Arg(OMe).AA and PEA-NTA-Agmatine.AA. Only PEA-NTA-Arg(OMe).AA was toxic at 1 mg/mL.

FIG. 3 is a graph showing percent viability of FL83B cells in the presence of polyarginine.

FIG. 4 is a graph showing percent viability of FL83B cells in the presence of invention polymer:DNA complexes containing GFP-encoding nucleic acid at various charge ratios and one each of Dharmafect®, Lipofectamine® and Superfect® as controls.

FIG. 5 is a graph summarizing flow cytometric data indicating percent of cells transfected with GFP-encoding DNA using various invention polymer complexes normalized to results with Dharmafect® transfection reagent.

FIG. 6 is a graph summarizing flow cytometric data indicating percent of GFP expression in Fl83B cells normalized to commercial transfection reagents.

FIG. 7 is a graph showing GFP fluorescence from three different cell types transfected by GFP plasmid DNA complexes with invention composition and with various commercial transfection reagents. Only the invention composition effectively transfected HeLa cells with GFP.

FIG. 8 is a graph showing percent expression of Sjorgen's syndrome B (SSB) gene in mouse liver cells transfected with complexes of siRNA with invention cationic PEA polymer and with commercial gene transfer agents.

FIG. 9 is a graph showing percent viability of FL83B cells transfected with of 100 nM DC03 (siRNA) complexed with different transfection reagents.

FIGS. 10A and 10B are 500 MHz ¹H NMR spectra in DMSO-d6 of FIG. 10A): PEA.H of Formula I; R²═OH and FIG. 10B): PEA-(OMe).HCl of Formula VI.

### A DETAILED DESCRIPTION OF THE INVENTION

Poly(ester-amide)s (PEAS) Poly(ester urethane)s PEURs and Poly(ester urea)s (PEUs) form a family of biodegradable polymers composed of ester and either amide, urethane or urea blocks in their backbones. PEAs have been studied widely for many years because these polymers combine the favorable properties of both polyesters and polyamides. When essential alpha-amino acids are used as building blocks these polymers have protein-like properties in addition to being biocompatible. For example, L-arginine is an α-amino acid present in the proteins of all life forms. The decarboxylated form of L-arginine, 4-aminobutyl guanidine, known as agmatine, belongs to the family of biogenic amines involved in many physiological functions.

Both arginine and agmatine carry a positive charge at physiological pH due to the strongly basic guanidino group and have a pKa value of about 12. (The ionized form of agmatine can be written as —NH—C(═NH₂⁺)—NH₂.) The invention utilizes arginine, agmatine and other cationic α-amino acids to provide cationic pendent groups in the PEAs and related PEURs and PEUs used in the invention compositions and methods. These pendent groups provide the strongly basic character necessary to neutralize and condense into soluble complexes that will penetrate cell membranes such nucleic acid sequences as DNA and RNA, which are negatively charged.

Accordingly, in one embodiment the invention provides a biodegradable gene transfer composition comprising at least one poly nucleic acid condensed into a soluble complex with a cationic polymer comprising at least one of the following:

a PEA polymer having a chemical formula described by general structural formula (I),

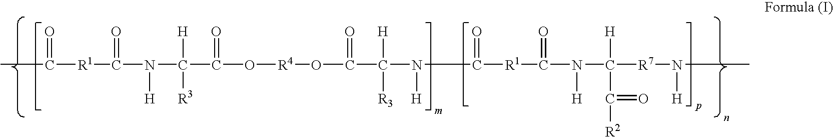

wherein n ranges from about 15 to about 150, m ranges about 0.1 to 0.9; p ranges from about 0.9 to 0.1;
R¹ is independently selected from the group consisting of (C₂-C₂₀) alkylene, (C₂-C₂₀) alkenylene, α,ω-bis(4-carboxyphenoxy)-(C₁-C₈) alkane, and α,ω-alkylene dicarboxylates of structural formula (II) and combinations thereof; wherein R⁵ in Formula (II) is independently selected from (C₂-C₁₂) alkylene, and (C₂-C₁₂) alkenylene, and R⁶ in Formula (II) is independently selected from the group consisting of (C₂-C₁₂) alkylene, (C₂-C₁₂) alkenylene, and (C₂-C₈) alkyloxy (C₂-C₂₀) alkylene,

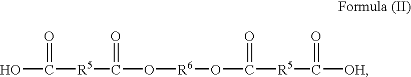

R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R¹—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and

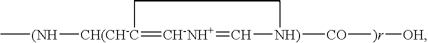

(polyhistidine), wherein r ranges from about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (III), and combinations thereof; and

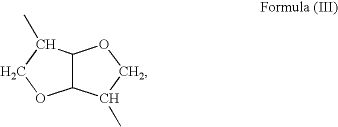

R⁷ is independently (C₂-C₂₀) alkyl or (C₂-C₂₀) alkenyl;

or a PEUR polymer having a chemical structure described by general structural formula (IV)

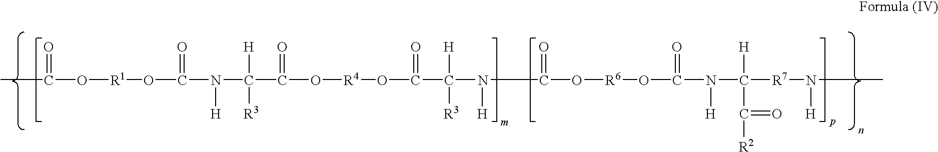

wherein n ranges from about 15 to about 150, m ranges from about 0.1 to 0.9; p ranges from about 0.9 to 0.1;
R¹ is independently selected from the group consisting of (C₂-C₂₀) alkylene, (C₂-C₂₀) alkenylene, α,ω-bis(4-carboxyphenoxy)-(C₁-C₈) alkane, and α,ω-alkylene dicarboxylates of structural formula (II) and combinations thereof; wherein R⁵ in Formula (II) is independently selected from (C₂-C₁₂) alkylene, and (C₂-C₁₂) alkenylene, and R⁶ in Formula (II) is independently selected from the group consisting of (C₂-C₁₂) alkylene, (C₂-C₁₂) alkenylene, and (C₂-C₈) alkyloxy (C₂-C₂₀) alkylene;
R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and

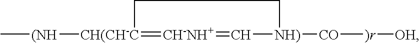

(polyhistidine), wherein r ranges from about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ and R⁶ are each independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof; and
R⁷ is independently (C₂-C₂₀) alkyl or (C₂-C₂₀) alkenyl;
   or a PEU polymer having a chemical formula described by general structural formula (V):

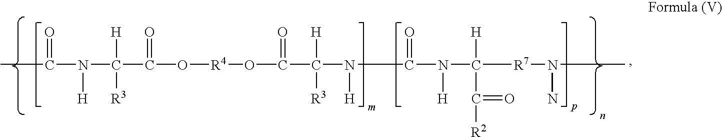

wherein n ranges from about 15 to about 150, m ranges about 0.1 to 0.9; p ranges from about 0.9 to 0.1;
R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and

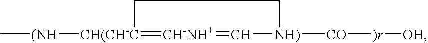

(polyhistidine), wherein r ranges form about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II); and
R⁷ is independently (C₂-C₂₀) alkyl or (C₂-C₂₀) alkenyl.

Preferred examples of R⁷ are (C₂-C₆) alkyl or (C₂-C₆) alkenyl, especially —(CH₂)₄—.

The examples of polymers synthesized for use in the present invention are PEAs of Formula I, wherein the C-terminus of L-lysine (R⁷═(CH₂)₄) in p monomer unit is covalently bound with either arginine methyl ester (VI) or agmatine (VII), and the guanidine pendent moieties are associated with acidic counter ions, for example from hydrochloric acid.

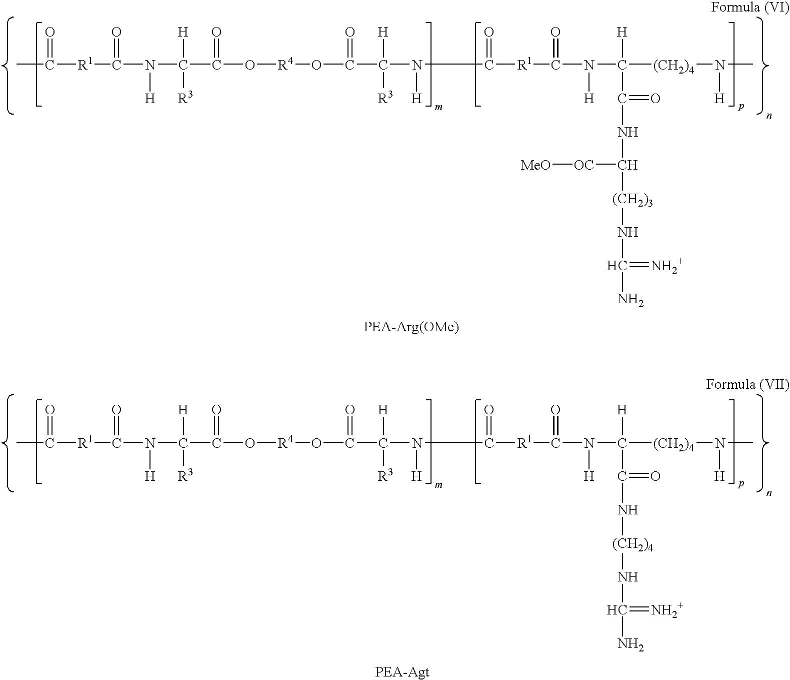

Methods of making alpha amino acid based PEAs and PEURs are disclosed in U.S. Pat. No. 6,503,538 B1, and method of preparing PEUs are described in (U.S. application Ser. No. 11/584,143). Procedures for conjugation of Arginine and Agmatine to PEA are described in Example 1 herein.

To vary the charge density along the macromolecule, guanidine derivatives can be bonded to the polymer through branched linkers. For example, the affinity ligand 6-amino-2-(bis-carboxymethylamino)-hexanoic acid (aminobutyl-, or AB-NTA, whose chemical structure is illustrated in formula VIII, has been used as a branched linker:

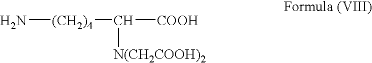

Prepared cationic PEAs were designated as PEA-NTA-Arg (formula IX) and PEA-NTA-Agt (formula X) and methods of their synthesis are disclosed below in Example 1.

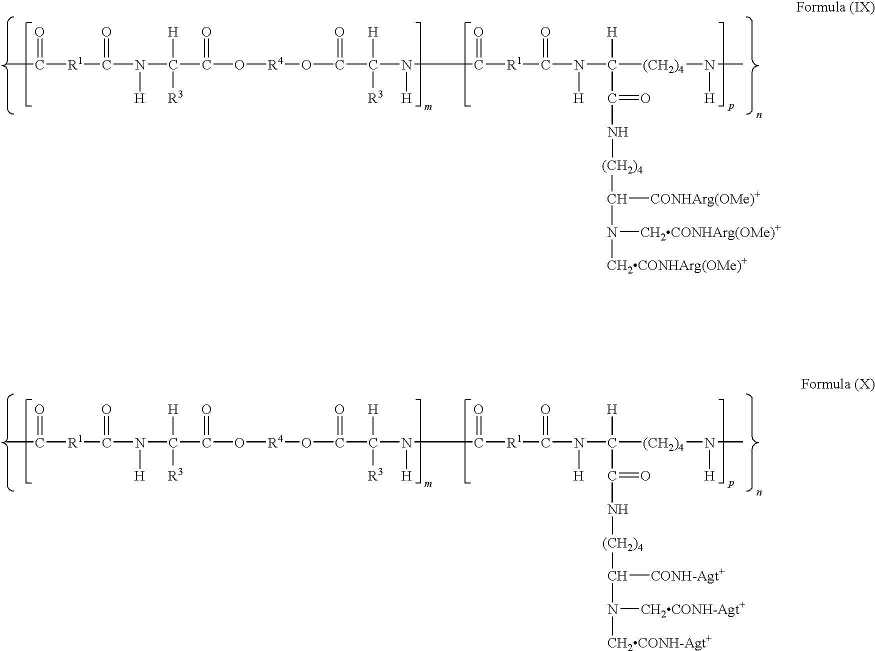

A general formula for PEA-NTA-conjugates is shown in Formula (XI):

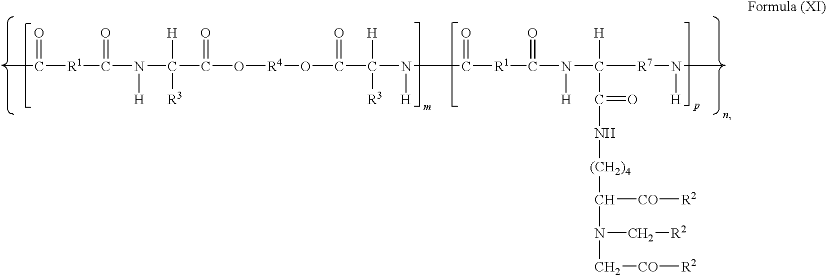

wherein n, m, p, R¹, R², R³, R⁴ and R⁷ are as defined above for PEAs of Formula (I).

The AB-NTA linker represents an α-N derivative of lysine. Additional examples of homologous linkers that can be used in fabrication of the cationic polymers contained in the invention gene transfer compositions are ornithine derivatives, whose chemical structures are described by general structural formula (XII) below.

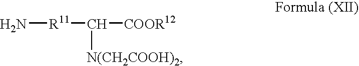

wherein R¹¹ is independently (C₂-C₈) alkylene, (C₂-C₈) alkenylene, and (C₂-C₈)alkyloxy (C₂-C₈) alkylene; for example (C₃-C₆) alkylene or (C₃-C₆) alkenylene; and R¹² is hydrogen, (C₁-C₁₂) alkyl, or (C₂-C₁₂) alkenyl. Preparation of such linkers and their conjugation with PEAs of structural formula (I) are exemplified in U.S. Publication No. 20070160622.

Additional examples of fabrication of cationic residues that can be used as the R² substituent in the PEA, PEUR and PEU polymers to increase charge density are made by a method of grafting arginine rich oligomers or commercially available low molecular weight cationic polyamino acids, such as oligoarginine, into the C-terminus of an amino acid in the p unit of the cationic PEA, PEUR or PEU polymers described herein. The grafting process can be carried out using a dicyclohexyl carbodiimide (DCC) type coupling, as shown in formula (XIII) wherein r is as defined above.

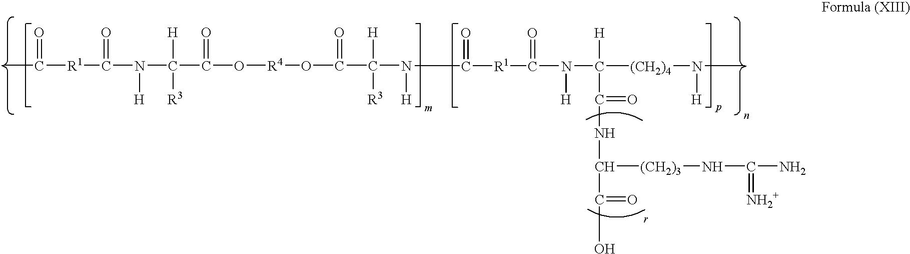

Other examples of cationic oligo- and polyamino acids that can be grafted to the invention polymers are polylysine, polyornithine, and polyhistidine.

In still another embodiment, the invention provides methods for transfecting a target cell by incubating the target cell in solution with the invention gene transfer composition comprising a poly nucleic acid condensed with the polymer therein under conditions and for a time to cause the composition to enter the target cells so as to transfect the target cell.

As used herein to describe the invention compositions and methods the terms “in solution” and “soluble complex” encompass the meanings commonly employed among biologists wherein particles suspended in a liquid are said to be in solution. The complex of the cationic polymer and poly nucleic acid in the invention compositions are condensed to form polymer particles in an aqueous environment as the charges on the polymer and the poly nucleic acid are neutralized. A suspension of such particles in a liquid is referred to herein as being in solution.

Suitable target cells for use in practicing the invention methods include, but are not limited to, mammalian cells, for example those belonging to tissues of a patient to be treated by expression of a poly nucleic acid delivered to the patient by administration of the invention composition. Suitable mammalian target cells include those of the nervous system (e.g., brain, spinal cord and peripheral nervous system cells), circulatory system cells (e.g., heart, vascular, and red and white blood cells), the digestive system (e.g., stomach and intestines), the respiratory system (e.g., the nose and the lungs), the reproductive system, the endocrine system (e.g., the liver, spleen, thyroid, and parathyroid), the skin, the muscles, or the connective tissue.

Alternatively, the target cells may be cancer cells derived from any organ or tissue, for example those belonging to tissues of a patient to be treated by expression of a poly nucleic acid delivered to the patient by administration of the invention composition. Alternatively still, the target cells can be those of a parasite, pathogen or virus infecting a patient or that can infect a subject. Thus, the invention gene transfer compositions are useful both in vitro, for studying interaction of a target cell with a desired poly nucleic acid expressed therein, and in vivo, for gene therapy applications in live subjects.

The structural formula for 4-methylene imidazolinium is as follows:

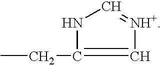

In certain embodiments, the polymer(s) in the composition can have one or more counter-ions associated with positively charged groups therein and/or one or more protecting groups bound to the polymer.

Known examples of counter-ions suitable to associate with the polymer in the invention composition are such counter-anions as Cl⁻, F⁻, Br⁻, CH₃COO⁻, CF₃COO⁻, CCl₃COO⁻, TosO⁻.

As used herein, the terms “water solubility” and “water soluble” as applied to the invention gene transfer compositions means the concentration of the composition per milliliter of deionized water at the saturation point of the composition therein. Water solubility will be different for each different polymer, but is determined by the balance of intermolecular forces between the solvent and solute and the entropy change that accompanies the solvation. Factors such as pH, temperature and pressure will alter this balance, thus changing the solubility. The solubility is also pH, temperature, and pressure dependent.

As generally defined, water soluble polymers can include truly soluble polymers to hydrogels (G. Swift, Polymer Degr. Stab. 59: (1998) 19-24). Invention compositions can be scarcely soluble (e.g., from about 0.01 mg/mL), or can be hygroscopic and when exposed to a humid atmosphere can take up water quickly to finally form a viscous solution in which composition/water ratio in solution can be varied infinitely.

The solubility of the polymers used in invention gene transfer compositions in deionized water at atmospheric pressure is in the range from about 0.01 mg/ml to 400 mg/ml at a temperature in the range from about 18° C. to about 55° C., preferably from about 22° C. to about 40° C. Quantitative solubility of the invention compositions can be visually estimated according to the method of Braun (D. Braun et al. in Praktikum der Makromolekularen Organischen Chemie, Alfred Huthig, Heidelberg, Germany, 1966). As is known to those of skill in the art, the Flory-Huggins solution theory is a theoretical model describing the solubility of polymers. The Hansen Solubility Parameters and the Hildebrand solubility parameters are empirical methods for the prediction of solubility. It is also possible to predict solubility from other physical constants, such as the enthalpy of fusion.

The addition of a low molecular weight electrolyte to a solution of a PEA, PEUR or PEUR polymer as described herein in deionized water can induce one of four responses. The electrolyte can cause chain contraction, chain expansion, aggregation through chelation (conformational transition), or precipitation (phase separation). The exact nature of the response will depend on various factors, such as the chemical structure, concentration, and molecular weight of the polymer and nature of added electrolyte. Nevertheless, invention gene transfer compositions can be soluble in various aqueous conditions, including those found in physiological conditions, such as blood, serum, tissue, and the like, or in water/alcohol solvent systems.

The water solubility of the invention compositions can also be characterized using such assays as ¹H NMR, ¹³C NMR, gel permeation chromatography, and DSC as is known in the art and as illustrated in the Examples herein.

All amino acids can exist as charged species, because of the terminal amino and carboxylate groups, but only a subset of amino acids have side chains that can, under suitable conditions, be charged. The term “cationic α-amino acid” as used herein to describe the polymers used in the invention compositions, means the R² groups are or contain amino acid residues whose side chains can function as weak acids—those not completely ionized when dissolved in water. The ionizable property is conferred upon such amino acid residues in the R² groups by the presence therein of an ionizable moiety consisting of a proton that is covalently bonded to a heteroatom, such as an oxygen, sulfur or nitrogen. Under suitable aqueous conditions, such as the proximity of another ionizable molecule or group, the ionizable proton dissociates from R² as the donating hydrogen ion, rendering the one or more amino acid residues in the R² substituent a base which can, in turn, accept a hydrogen ion. Dissociation of the proton from the acid form, or its acceptance by the base form is strongly dependent upon the pH of the aqueous milieu. Ionization degree is also environmentally sensitive, being dependent upon the temperature and ionic strength of the aqueous milieu as well as upon the micro-environment of the ionizable group within the polymer.

Thus, the term “cationic α-amino acid” as used herein to describe certain of the polymers in invention gene transfer compositions, means the amino acid residues in R² groups of amino acid residues therein can form positive ions under suitable ambient aqueous or solvent conditions, especially under physiological conditions, such as in blood and tissue. Counter-ions of such positive amino acids can be as described above.

As used herein, the term “residue of a di-acid” means that portion of a dicarboxylic-acid that excludes the two carboxyl groups of the di-acid, which portion is incorporated into the backbone of the invention polymer compositions. As used herein, the term “residue of a diol” means that portion of a diol that excludes the two hydroxyl groups thereof at the points the residue is incorporated into the backbone of the invention polymer compositions. The corresponding di-acid or diol containing the “residue” thereof is used in synthesis of the invention gene transfer compositions.

The di-aryl sulfonic acid salts of diesters of α-amino acid and diol can be prepared by admixing α-amino acid, e.g., p-aryl sulfonic acid monohydrate, and diol in toluene, heating to reflux temperature, until water evolution has ceased, then cooling.

Saturated di-p-nitrophenyl esters of dicarboxylic acid and saturated di-p-toluene sulfonic acid salts of bis-α-amino acid esters can be prepared as described in U.S. Pat. No. 6,503,538 B1.

PEA, PEUR and PEU polymers of Formulas (I, IV and V) containing cationic α-amino acids can be prepared using protective group chemistry. Protected monomers will be de-protected either prior to APC or after polymer work-up. Suitable protective reagents and reaction conditions used in protective group chemistry can be found, e.g. in Protective Groups in Organic Chemistry, Third Edition, Greene and Wuts, Wiley & Sons, Inc. (1999), the content of which is incorporated herein by reference in its entirety.

The poly nucleic acid in the invention compositions can include deoxyribonucleic acid (DNA), ribonucleic acid (RNA), double stranded DNA, double stranded RNA, duplex DNA/RNA, antisense poly nucleic acids, functional RNA or a combination thereof. In one embodiment, the poly nucleic acid can be RNA. In another embodiment, the poly nucleic acid can be DNA. In another embodiment, the poly nucleic acid can be an antisense poly nucleic acid. In another embodiment the poly nucleic acid can be a sense poly nucleic acid. In another embodiment, the poly nucleic acid can include at least one nucleotide analog. In another embodiment, the poly nucleic acid can include a phosphodiester linked 3′-5′ and 5′-3′ poly nucleic acid backbone. Alternatively, the poly nucleic acid can include non-phosphodiester conjugations, such as phosphothioate type, phosphoramidate and peptide-nucleotide backbones. In another embodiment, moieties can be linked to the backbone sugars of the poly nucleic acid. Methods of creating such conjugations are well known to those of skill in the art.

The poly nucleic acid can be a single-stranded poly nucleic acid or a double-stranded poly nucleic acid. The poly nucleic acid can have any suitable length. Specifically, the poly nucleic acid can be about 2 to about 5,000 nucleotides in length, inclusive; about 2 to about 1000 nucleotides in length, inclusive; about 2 to about 100 nucleotides in length, inclusive; or about 2 to about 10 nucleotides in length, inclusive.

An antisense poly nucleic acid is typically a poly nucleic acid that is complimentary to an mRNA that encodes a target protein. For example, the mRNA can encode a cancer promoting protein i.e., the product of an oncogene. The antisense poly nucleic acid is complimentary to the single-stranded mRNA and will form a duplex and thereby inhibit expression of the target gene, i.e., will inhibit expression of the oncogene. The antisense poly nucleic acids of the invention can form a duplex with the mRNA encoding a target protein and will disallow expression of the target protein.

A “functional RNA” refers to a ribozyme or other RNA that is not translated.

A “poly nucleic acid decoy” is a poly nucleic acid that inhibits the activity of a cellular factor upon binding of the cellular factor to the poly nucleic acid decoy. The poly nucleic acid decoy contains the binding site for the cellular factor. Examples of such cellular factors include, but are not limited to, transcription factors, polymerases and ribosomes. An example of a poly nucleic acid decoy for use as a transcription factor decoy will be a double-stranded poly nucleic acid containing the binding site for the transcription factor. Alternatively, the poly nucleic acid decoy for a transcription factor can be a single-stranded nucleic acid that hybridizes to itself to form a snap-back duplex containing the binding site for the target transcription factor. An example of a transcription factor decoy is the E2F decoy. E2F plays a role in transcription of genes that are involved with cell-cycle regulation and that cause cells to proliferate. Controlling E2F allows regulation of cellular proliferation. For example, after injury (e.g., angioplasty, surgery, stenting) smooth muscle cells proliferate in response to the injury. Proliferation may cause restenosis of the treated area (closure of an artery through cellular proliferation). Therefore, modulation of E2F activity allows control of cell proliferation and can be used to decrease proliferation and avoid closure of an artery. Examples of other such poly nucleic acid decoys and target proteins include, but are not limited to, promoter sequences for inhibiting polymerases and ribosome binding sequences for inhibiting ribosomes. It is understood that the invention includes poly nucleic acid decoys constructed to inhibit any target cellular factor.

A “gene therapy agent” refers to an agent that causes expression of a gene product in a target cell through introduction of a gene into the target cell followed by expression of the gene product. An example of such a gene therapy agent would be a genetic construct that causes expression of a protein, when introduced into a cell, such as a DNA vector. Alternatively, a gene therapy agent can decrease expression of a gene in a target cell. An example of such a gene therapy agent would be the introduction of a poly nucleic acid segment into a cell that would integrate into a target gene or otherwise disrupt expression of the gene. Examples of such agents include poly nucleic acids that are able to disrupt a gene through homologous recombination. Methods of introducing and disrupting genes within cells are well known to those of skill in the art and as described herein.

In one embodiment, the poly nucleic acid can be synthesized according to commonly known chemical methods. In another embodiment, the poly nucleic acid can be obtained from a commercial supplier. The poly nucleic acid can include, but is not limited to, at least one nucleotide analog, such as bromo derivatives, azido derivatives, fluorescent derivatives or a combination thereof. Nucleotide analogs are well known to those of skill in the art. The poly nucleic acid can include a chain terminator. The poly nucleic acid can also be used, e.g., as a cross-linking reagent or a fluorescent tag. Many common conjugations can be employed to couple a poly nucleic acid to another moiety, e.g., phosphate, hydroxyl, etc. Additionally, a moiety may be linked to the poly nucleic acid through a nucleotide analog incorporated into the poly nucleic acid. In another embodiment, the poly nucleic acid can include a phosphodiester linked 3′-5′ and 5′-3′ poly nucleic acid backbone. Alternatively, the poly nucleic acid can include non-phosphodiester conjugations, such as phosphothioate type, phosphoramidate and peptide-nucleotide backbones. In another embodiment, moieties can be linked to the backbone sugars of the poly nucleic acid. Methods of creating such conjugations are well known to those of skill in the art.

The condensed polymer:poly nucleic acid can degrade in vitro in contact with an enzyme, such as α-chymotrypsin, or when injected in vivo to provide time release of a suitable and effective amount of the poly nucleic acid. Any suitable and effective period of time can be chosen. Typically, the suitable and effective amount of poly nucleic acid can be released in about twenty-four hours in about 2 days or in about seven days. Factors that typically affect the length of time over which the poly nucleic acid is released from the invention composition include, e.g., the nature and amount of polymer, the nature, size and amount of poly nucleic acid, the pH, temperature and electrolyte or enzyme content of the environment into which the composition is introduced.

Any suitable size of PEA, PEUR or PEU polymer of Formula (I, IV or V) can be employed in the invention gene deliver compositions. For example, the polymer can have a size of less than about 1×10⁻⁴ meters, less than about 1×10⁻⁵ meters, less than about 1×10⁻⁶ meters, less than about 1×10⁻⁷ meters, less than about 1×10⁻⁸ meters, or less than about 1×10⁻⁹ meters.

The invention gene transfer compositions and methods encompass the use and delivery to target cells of RNA and DNA of all types, including poly nucleic acids, poly nucleic acids and poly nucleic acids. More specifically, the nucleic acid can be any DNA or RNA. DNA includes a plasmid for expression of a gene contained therein, such as a gene encoding a therapeutic molecule. RNA includes messenger (mRNA), transfer (tRNA), ribosomal (rRNA), and interfering (iRNA). Interfering RNA is any RNA involved in post-transcriptional gene silencing, which includes, but is not limited to, double stranded RNA (dsRNA), small interfering RNA (siRNA), and microRNA (miRNA) that are comprised of sense and antisense strands. In the mechanism of RNA interference, dsRNA enters a cell and is digested to 21-23 nucleotide siRNAs by the enzyme DICER therein. Successive cleavage events degrade the RNA to 19-21 nucleotides known as siRNA. The siRNA antisense strand binds a nuclease complex to form the RNA-induced silencing complex, or RISC. Activated RISC targets the homologous transcript by base pairing interactions and cleaves the mRNA, thereby suppressing expression of the target gene. Recent evidence suggests that the machinery is largely identical for miRNA (Cullen, B. R. (2004) Virus Res. 102:3). In this way, iRNA, once condensed with the polymer, can be delivered into a cell by phago- or pino-cytosis and released to enter the cell's normal biological processing pathway as a means of suppressing expression of a target gene.

The emerging sequence-specific inhibitors of gene expression, small interfering RNAs (siRNAs), have great therapeutic potential; however, development of such molecules as therapeutic agents is hampered by rapid degradation of siRNA in vivo. Therefore a key requirement for success in therapeutic use of siRNA is the protection of the gene silencing nucleic acid. In the present invention, such protection for siRNA is provided by condensation of the poly nucleic acid molecule with the cationic PEA, PEUR or PEU polymers described herein.

For, example, in fabrication of the invention composition for delivery of the antisense strand of iRNA, the antisense strand of negatively charged iRNA is condensed with the cationic polymer. The ds RNA is condensed with the carrier polymer. Alternatively, the sense strand can be condensed with one polymer chain and the antisense strand with another polymer chain. In either case, double stranded RNA, released from the invention composition during biodegradation of the polymer, and the antisense strand, freed from the sense strand, would enter the normal biological pathway for iRNA.

To illustrate the invention, PEA polymers with a pendent cationic guanidine group were prepared as described in Example 1 herein and used to condense plasmid DNA or siRNA sufficiently for the invention gene transfer compositions to easily enter mouse hepatocyte liver cells in vitro. Physico-chemical tests (gel electrophoresis, fluorescence, green fluorescent protein expression assays) have confirmed successful cell transfection and expression of the poly nucleic acid in the invention composition. GFP expression assays were performed to evaluate transfection efficiency of the invention gene transfer compositions as compared with commercial gene transfer agents: Lipofectamine®, Dharmafect®, and Superfect®.

More particularly, Arginine- or Agmatine-conjugated poly(ester-amide)s of formulas (VI, VII, IX, X) were evaluated for efficiency as a non-viral gene transfer agent to effect transfection of a target cell, for example to be used in gene therapy.

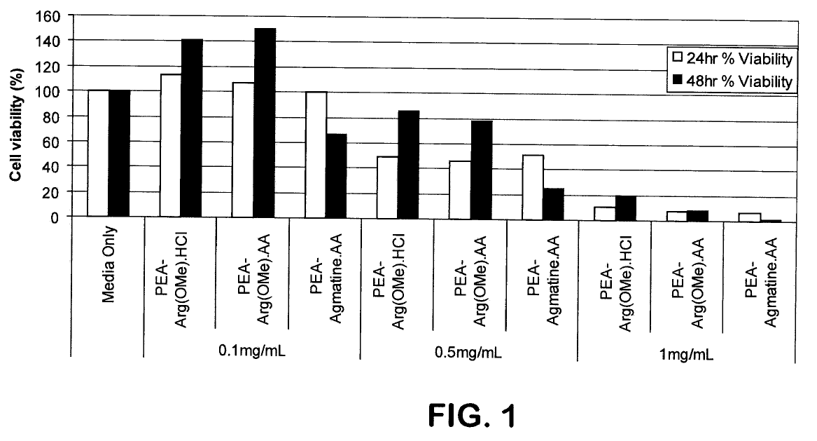

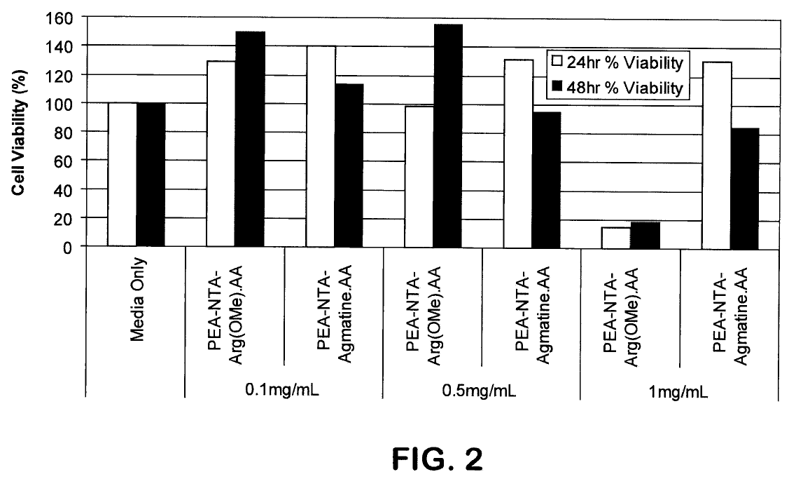

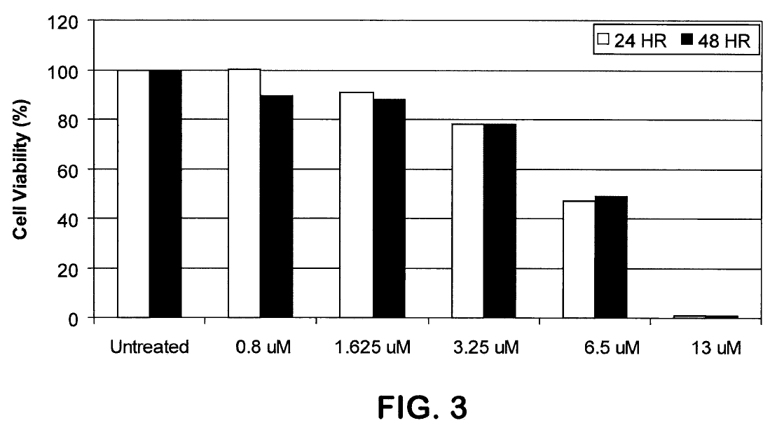

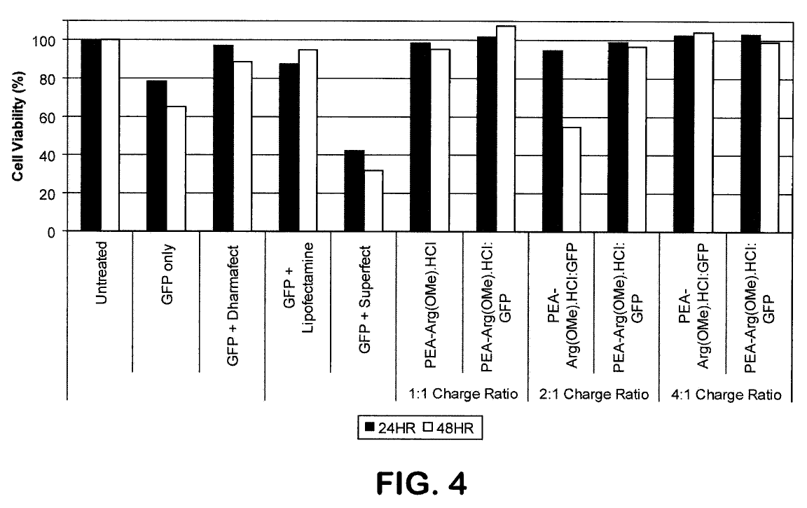

Because the ratio of polymer to poly nucleic acid used in the invention methods to effect condensation may in some cases be greater than in prior art gene transfer agents, cytotoxicity of the polymers was assayed by incubating the polymers with mouse liver FL83B cells. Cytotoxicity was measured at 24 and 48 hours using a standard luminometer cell proliferation assay. As shown by the data from this cell viability experiment as summarized in FIGS. 1-3, only PEA-Agmatine.AA showed toxicity at 0.1 mg/mL concentration. All other invention cationic polymers are not toxic at 0.1 mg/mL concentration. PEA-NTA-Agmatine.AA was not toxic even at 1 mg/mL concentrations. Overall, all PEA conjugates were less toxic than commercial polyarginine at similar concentrations (13 uM polyarginine is 0.2 mg/ml).) When compared with commercially available transfection reagents used in these studies as controls, FL83B cells incubated with invention gene transfer compositions (i.e., polymer: DNA complexes) were as viable as with the best commercially available transfection reagents and were generally 60% more viable than with Superfect® (FIG. 4).

The term “charge ratio” as used to describe the invention gene transfer compositions means the ratio of positive polymer charge to negative poly nucleic acid charge. For each of the invention compositions made to illustrate the invention, described, the total number of positive charges was calculated based on % of guanidinium load per polymer, which was estimated by ¹H NMR data. For both DNA and siRNA, the number of negative charges was based on two negative charges per base pair and calculated as the total number of charges per mass. The ratio of positive polymer charge to negative poly nucleic acid charge was determined to be the charge ratio as shown in Table 1 and 2 below.

Gel retardation assays and zeta potential of condensate of the invention composition in aqueous suspension were used to confirm that the positively charged PEA polymers were able to neutralize negatively charged plasmid DNA to form a compact complex suitable for use in transfection of a target cell. The siRNA was formulated with PEA-Arg(OMe) at charge ratios of 1:1, 2:1, and 4:1 polymer to siRNA. Although at a 1:1 charge ratio, unbound siRNA was observed in the agarose gel, at charge ratios of 2:1, 4:1, and 6:1, the siRNA was fully complexed with the polymer as shown in Table 2 below. Thus, it has been discovered that the cationic PEA, PEUR and PEU polymers described herein have affinity to complex a poly nucleic acid and that the overall charge of the condensate particle formed changes according to excess of the cationic polymer.

TABLE 1
COMPLEXES PEA_I_Arg(Ome)HCl: GFP

| | | | | |
|---|---|---|---|---|
| PEA Only | GFP | 1:1 | 2:1 | 4:1 |

Zeta Potentials (Charge Ratios, mV)

| | | | | | |
|---|---|---|---|---|---|
| 20 mM HEPES | | | | | |
| Buffer pH 7.4 | | | | | |
| −39 | 41 | −17 | −43 | 29 | 48 |

DLS (diameter in nm)

| | | | | | |
|---|---|---|---|---|---|
| HEPES | | | | | |
| 838.7 | 139.8 | 108.8 | 97.7 | 111.8 | 116.7 |

PDI

| | | | | | |
|---|---|---|---|---|---|
| 0.728 | 0.161 | 0.192 | 0.172 | 0.216 | 0.222 |

TABLE 2
COMPLEXES PEA-Arg(OMe)HCl: siRNA

| | | | | |
|---|---|---|---|---|
| PEA Only | siRNA (DC-03) | 1:1 | 2:1 | 4:1 |

Zeta Potentials (Charge Ratios, mV)

| | | | | | |
|---|---|---|---|---|---|
| 20 mM HEPES | | | | | |
| Buffer pH 7.4 | | | | | |
| −7.98 | 47.6 | −6.95 | −36.5 | 46.2 | 49 |

DLS (diameter in nm)

| | | | | | |
|---|---|---|---|---|---|
| HEPES | | | | | |
| 81.13 | 55.63 | 26.29 | 140 | 110.4 | 84.99 |

PDI

| | | | | | |
|---|---|---|---|---|---|
| 1 | 0.494 | 0.735 | 0.207 | 0.146 | 0.26 |

To illustrate expression of the poly nucleic acid cargo in target cells, invention compositions comprising complexes of cationic PEA and siRNAs against Sjorgen's syndrome B (SSB) made in serum free media were used to transfect the FL83B cells by incubation of the cells with the invention compositions in serum free media, as described herein in Example 3. Those of skill in the art will understand that any transfection conditions known in the art as suitable for use in cell transfection may also be used.

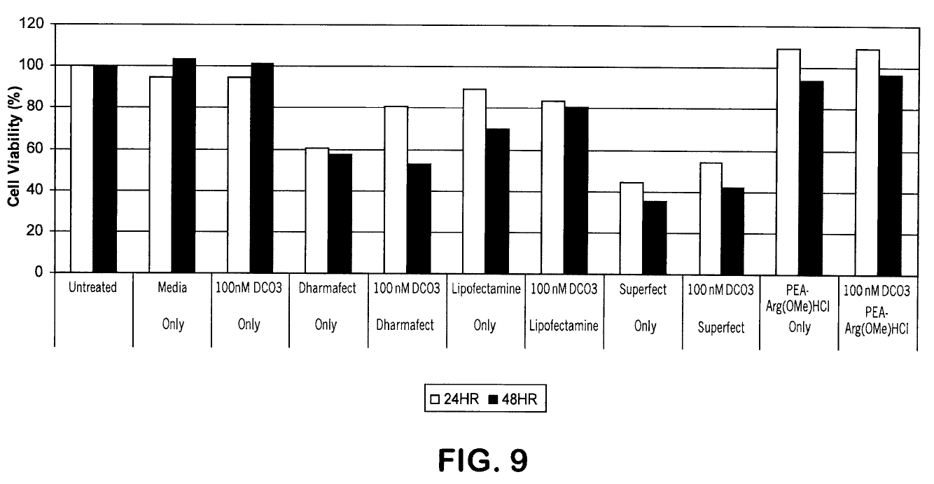

When the target cells were harvested, RNA was isolated, and gene expression was measured by quantitative PCR using standard methods, it was discovered (FIG. 9) that transfection of siRNA complexed to PEA-Arg(OMe).HCl or to Dharmafect® resulted in approximately equivalent (i.e., 70%) down regulation of SSB expression in the target cells.

The following Examples are meant to illustrate, and not to limit, the invention.

### EXAMPLE 1

### A. Materials Characterization

The chemical structure of monomers and polymers were characterized by standard chemical methods; NMR spectra were recorded by a Bruker AMX-500 spectrometer (Numega R. Labs Inc. San Diego, Calif.) operating at 500 MHz for ¹H NMR spectroscopy. Deuterated solvents CDCl₃ or DMSO-d₆ (Cambridge Isotope Laboratories, Inc., Andover, Mass.) were used with tetramethylsilane (TMS) as internal standard.

Melting points of synthesized monomers were determined on an automatic Mettler-Toledo FP62 Melting Point Apparatus (Columbus, Ohio). The number and weight average molecular weights (Mw and Mn) and molecular weight distribution of synthesized polymers were determined by Model 515 gel permeation chromatography (Waters Associates Inc. Milford, Mass.) equipped with a high pressure liquid chromatographic pump, a Waters 2414 refractory index detector. 0.1% of LiCl solution in N,N-dimethylacetamide (DMAc) was used as eluent (1.0 mL/min). Two Styragel® HR 5E DMF type columns (Waters) were connected and calibrated with polystyrene standards.

### B. General Procedure for Activation of PEA (PEA-OSu)

5.0 g (2.7 mmol, weight average Mw=65 kDa, GPC(PS)) of PEA polymer (PEA-OSu) (Formula I; wherein R¹═(CH₂)₈; R²═OH; and R³═CH₂CH(CH₃)₂, R⁴═(CH₂)₆, R⁷═(CH₂)₄, synthesized according to methods in U.S. Pat. No. 6,503,538 B1), was dissolved in 50 mL of dry dimethylformamide (DMF). Then 0.615 g of dicyclohexyl carbodiimide (DCC, 2.98 mmol) and 0.374 g of N-hydroxysuccinimide (HOSu, 3.25 mmol) were added and the mixture was stirred under argon for about. 12 hours at room temperature. Formed residue was removed by filtering through 0.45 micron pore size frit (PTFE syringe filters). A solution of activated PEA-OSu was kept under argon for further conjugations. From 80%-100% of the PEA was activated by conjugation with OSu, as determined by ¹H-NMR analysis.

### C. Synthesis of PEA-Arg(OMe) Conjugate (Formula VI)

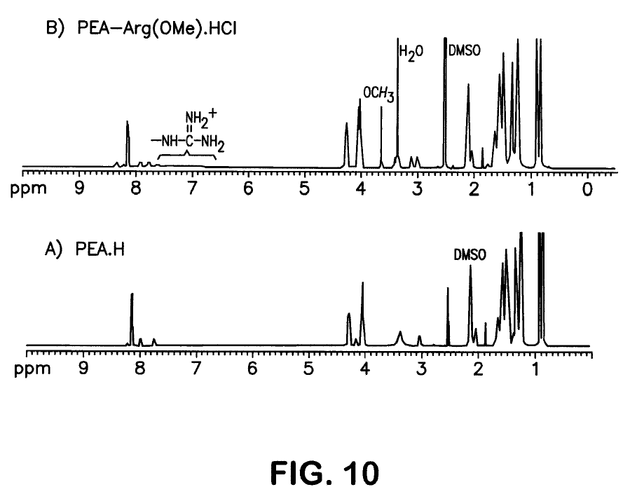

Into the previously prepared solution of activated ester of PEA-OSu (containing 5.3 g, 2.7 mmol polymer), 0.849 g of L-arginine methyl ester dihydrochloride (3.25 mmol), 1.134 mL of N,N-diisopropyl ethylamine (DIPEA, 6.50 mmol) and 500 mL of DMF were added under argon. The resulting heterogeneous mixture was stirred at room temperature for about 24 hours. The PEA-Arg(OMe) polymer conjugate so formed was precipitated into 5 L of ethyl acetate with 3% by volume of acetic acid. The precipitate was rinsed again with ethyl acetate and dried with paper towels. The collected polymer precipitate was re-dissolved in ethanol (5.0 g into 50 mL), transferred into dialysis bags with a molecular weight cut-off of 3500 Da and dialyzed in DI water. A final dialyzed solution was freeze-dried and analyzed by ¹H-NMR, GPC and DLS for zeta potential and particle size. From 60%-90% of the polymer was converted to Arg(OMe) as determined by ¹H-NMR (see FIG. 10). The yield of product PEA-Arg(OMe) conjugate after purification ranged from 80-90% with weight average molecular weight (Mw) of approximately 70 kDa, (as determined by GPC, PS).

### D. Synthesis of PEA-Agmatine Conjugate, (Formula VII)

A suspension of 0.5 g of Agmatine sulfate (2.19 mmol) and 0.21 g of sodium hydroxide (8.76 mmol) was stirred in 10 mL of DMF for 12 h at room temperature and the solution formed was filtered through 0.45 micron pore site frit (PTFE syringe filters). 5 mL of resulting agmatine (free amine form, 18.7 mmol) in 5% (weight/volume) DMF and 0.53 mL of acetic acid (9.3 mmol) in 21.6 mL of DMF was added to the previously prepared solution of activated PEA-OSu (3.0 g, 15.5 mmol). Formed heterogeneous mixture was stirred at room temperature for ca. 24 h under argon. PEA-agmatine polymer conjugate was precipitated into 2.5 L of ethyl acetate. The precipitate was rinsed with ethyl acetate and dried with paper towels. The collected polymer conjugate was re-dissolved in ethanol (3.0 g, 100 mL). Dissolved polymer was transferred into dialysis bags with a molecular weight cut-off of 3500 Da and dialyzed in DI water. Dialyzed product PEA-Agmatine conjugate was freeze-dried and analyzed by ¹H-NMR, GPC, DSC, and DLS for zeta potential and particle size. The agmatine load to polymer ranged from 50-60% as determined by NMR. The reaction yield after purification ranged from 70-80% with weight average molecular weight (Mw) of approximately 70 kDa, (GPC, PS).

### E. General Procedure for Activation of PEA.I.NTA (PEA-NTA-OSu)

5.0 g (2.40 mmol, weight average Mw=77 kDa, GPC(PS)) of PEA polymer (Formula I, wherein R¹═(CH₂)₈; R²═linker of formula VIII; and R³═CH₂CH(CH₃)₂, R⁴═(CH₂)₆, R⁷═(CH₂)₄,) was dissolved in 50 mL of dry dimethylformamide (DMF) under argon. Then, 1.535 g of dicyclohexylcarbodiimide (DCC, 7.44 mmol) and 0.884 g of N-hydroxysuccinimide (HOSu, 7.68 mmol) were added and the mixture was stirred for about 8 hours at room temperature. Formed residue was removed by filtering through 0.45 micron pore size frit (PTFE syringe filters). A solution of PEA-OSu conjugate was collected into a round bottom flask and kept under argon. A sample polymer solution was analyzed by ¹H-NMR for OSu load, which ranged from 80-100%.

### F. Synthesis of PEA-NTA-Arg(OMe) Conjugate (Formula IX)

Into the solution of activated ester of PEA-NTA-OSu (5.7 g, 2.4 mmol) in DMF were added 2.08 g of L-arginine methyl ester dihydrochloride (Arg(OMe), 7.96 mmol), 2.77 mL of N,N-diisopropylethylamine (DIPEA, 7.2 mmol) and 500 mL of DMF. The resulting heterogeneous mixture was stirred at room temperature for about 24 hours. The PEA-NTA-Arg(OMe) polymer conjugate was precipitated into 5 L of ethyl acetate with 1% v/v of acetic acid. The precipitate was rinsed with ethyl acetate and dried with paper towels. The collected polymer conjugate was redissolved in ethanol (5.0 g in 50 mL), diluted with 20 mL water and transferred into dialysis bags with a molecular weight cut-off of 3500 Da. The polymer was dialyzed in deionized water for two days and then was filtered and freeze-dried. The product was analyzed by ¹H-NMR, GPC, DSC, and DLS for zeta potential and particle size. The Arg(OMe) load to polymer ranged from 50-70%, as determined by ¹H-NMR. Product yield after purification ranged from 80-90%. Weight average molecular weight (Mw) was in a range of 155 to 160 kDa, (GPC, PS).

### G. Synthesis of PEA-NTA-Agmatine Conjugate (Formula X)

Into the activated ester of PEA-NTA-OSu (4.55 g, 19.1 mmol) in a round bottom flask, the following reagents were added: 18.43 mL of Agmatine (68.8 mmol) in 5% (weight/volume) of DMF, 1.97 mL of acetic acid (34.4 mmol) and 20.5 mL of DMF. Amine form of agmatine solution was prepared as follows: 0.5 g of agmatine sulfate (2.19 mmol) and 0.21 g of sodium hydroxide (8.76 mmol) were dispersed in 10 mL DMF solution and stirred overnight (about 12 hrs). The solution was filtered through A 0.45 micron pore size frit (PTFE syringe filters).

The solution of the amine form of agmatine was added into the solution in the round bottomed flask and a resulting suspension was stirred at room temperature for about 24 h. The resulting PEA-NTA-Agmatine polymer conjugate was precipitated into 2.5 L of ethyl acetate. The precipitate was rinsed with ethyl acetate and dried with paper towel. The collected polymer was dissolved in ethanol (3.0 g, 100 mL) and transferred into a dialysis bag with a molecular weight cut-off of 3500 Da. The polymer conjugate was dialyzed in 3.5 L of DI water, solution was filtered and lyophilized. The product PEA-NTA-Agmatine conjugate (Formula X) was analyzed by ¹H-NMR, GPC, DSC, and DLS for zeta potential and particle size. The Agmatine load to polymer ranged from 80-90% by ¹H-NMR. The reaction yield after purification ranged from 50-60%. Weight average molecular weight (Mw) was in a range of 150 to 180 kDa, (GPC, PS).

### EXAMPLE 2

### A. Materials

Ethidium bromide was purchased from Sigma (St. Louis, Mo.), phosphate-buffered saline (PBS, pH 7.4) was purchased from Cellgro (Herndon, Va.), HEPES (Calbiochem, San Diego, Calif.), the DNA size marker TRACK IT™ (Invitrogen, Carlsbad, Calif.), Superfect® (Qiagen, Valencia, Calif.), Lipofectamine® (Invitrogen, Carlsbad, Calif.), and Dharmafect® (Dharmacon, Lafayette, Colo.), were purchased from commercial sources. Other chemicals and reagents, if not otherwise specified, were purchased from Sigma (St. Louis, Mo.).

### B. Preparation of Plasmid DNA

Plasmid DNA was prepared using a Qiagen endotoxin-free plasmid maxi-prep kit according to the supplier's protocol. The quantity and quality of the purified plasmid DNA was assessed by spectrophotometric analysis at 260 nm as well as by electrophoresis on a 1% agarose gel. Purified plasmid DNAs were resuspended in 10 mM Tris-Cl; pH 8.5 and frozen in aliquots.

### C. Cell Culture

Mouse liver cells FL83B, were obtained from American Type Culture Collection (ATCC, Manassas, Va.). The FL83B cells were grown as recommended at 37° C. in 5% CO₂ in Kaighn's F12K complete media supplemented with 10% fetal bovine serum.

### D. Preparation of Invention PEA Stock Suspension

Polymers prepared in Example 1 above were dissolved at 100 mg/mL in 200 proof ethanol. A 10 mg/mL polymer suspension was made by adding 100 μL of 100 mg/mL of the various polymers to 900 μL water. Ethanol in the polymer suspension was removed partially by rotary evaporator. The suspension was returned to its original volume by the addition of water. The 10 mg/mL polymer suspension was used for the following experiments or a further dilution was made in water to 1 mg/mL.

### E. Assessment of Polymer Cytotoxicity

The biocompatibility of the invention PEA polymers was tested in mouse hepatocyte FL83B cells. The following invention PEA polymers were used for the cytotoxicity study: PEA-Arg(OMe).HCl, PEA-Arg(Ome).AA, PEA-NTA-Arg(OMe).AA, PEA-Agt.AA, PEA-NTA-Agt.AA (of formulas VI, VII, IX, X, where R¹═(CH₂)₈; R³═CH₂CH(CH₃)₂, R⁴═(CH₂)₆; AA—acetic acid; p=0.75; m=0.25) and Polyarginine hydrochloride (Mol wt 5,000-15,000 Sigma St. Louis, Mo.).

Polymers were added to FL83B cells and cytotoxicity was measured at 24 and 48 hours using ViaLight® Plus Cell Proliferation and Cytotoxicity BioAssay Kit (Cambrex, Rockland, Me.). 100 mg/mL polymer was added to cell culture media supplemented with 10% fetal bovine serum to a final concentration of 0.1 mg/mL, 0.5 mg/mL, or 1 mg/mL. The medium was removed and cell lysis reagent added. After 10 minutes, 100 μl of the cell lysate was transferred to a white walled luminometer plate. 100 μl of ATP Monitoring Reagent Plus was added to each well. Plates were incubated for 2 min and then read in a luminometer. The % viability data are expressed as percent viability normalized to the control as calculated by dividing the sample relative luminescence by control relative luminescence×100.

As shown by the data from this experiment as summarized in FIGS. 1-3, only PEA-Agmatine.AA showed toxicity at 0.1 mg/mL concentration. All other invention cationic polymers are not toxic at 0.1 mg/mL concentration. PEA-NTA-Agmatine.AA was not toxic even at 1 mg/mL concentrations. Overall, all PEA conjugates were less toxic than commercial polyarginine at similar concentrations (13 uM polyarginine is 0.2 mg/ml).

### F. Definition and Measurement of Charge Ratio

For each of the polymers described, the total number of positive charges was calculated based on % of guanidinium load per polymer, which was estimated by the ¹H NMR. For both DNA and siRNA, the number of negative charges was based on two negative charges per base pair and calculated as the total number of charges per mass. The ratio of positive polymer charge to negative poly nucleic acid charge was determined to be the charge ratio and entered as categories in Table 1 and 2.

Formation of the polymer: DNA complex was also confirmed by zeta potential measured on Dynamic Light Scattering (DLS) equipment (Zetasizer Nano ZS equipped with Dispersion Technology Software 5.00, Malvern Instruments Ltd, Worcestershire, UK). Results can be seen in Tables 1 and 2 herein. PEA-Arg(OMe) suspension was complexed with GFP plasmid. The sample was brought up to 1 mL in 20 mM HEPES buffer pH 7.4, then entire 1 mL volume was loaded into a disposable capillary cell (Malvern, DTS1060) according to product protocol.

### G. Cytotoxicity of Polymer: DNA Complex

Cytotoxicity of the polymer: DNA complex was measured as described in previous example for polymer only. Briefly, polymer: DNA complexes were made by adding a volume of 10 mg/mL polymer suspension to a volume of 1 mg/mL GFP plasmid in serum free media at charge ratios of 1:1, 2:1, and 4:1 for a final concentration of 1 μg GFP plasmid DNA for each well in a 24 well plate. The suspensions were immediately vortexed for several seconds after mixing the solutions, and then allowed to equilibrate at ambient conditions for 40 minutes. These complexes were added to cells for 18-24 hours at 37° C. under 5% CO₂ The cell culture media including the polymer: DNA complex solution was removed and replaced with fresh media. Cytotoxicity of the polymer: DNA complex was measured at 24 and 48 hours by Vialight® assay (East Rutherford, N.J.). As shown by the data summarized in FIG. 4, FL83B cells in the presence of polymer: DNA complexes were as viable as with the best commercially available transfection reagents and were generally 60% more viable than with Superfect.

### [H] Transfection with Green Fluorescent Protein Plasmid (GFP)

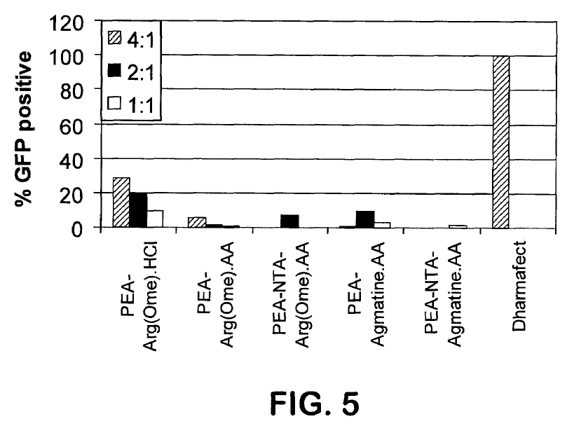

DNA was complexed with PEA-Arg(OMe).HCl (formula VI) at charge ratios of 1:1, 2:1, and 4:1 polymer to DNA as described above. Plasmid DNA expressing green fluorescent protein was used so that transfection efficiency could be monitored microscopically. Polymer: DNA complexes were made in 20 mM HEPES buffer or in serum free cell culture media. The polymer: DNA complex was confirmed by running an agarose gel retardation assay. Briefly, polymer: DNA complexes formed using the above protocol were analyzed by electrophoresis on a 1% agarose gel stained with ethidium bromide in TAE (Tris acetate EDTA) buffer at 100 V for 15-20 min. DNA was visualized by UV illumination. Free DNA will migrate through the gel and can be visualized with ethidium bromide staining whereas polymer:DNA condensates will not migrate through the gel. FIG. 5 shows polymer:DNA condensates at four charge ratios. At a 0.5:1 and 1:1 charge ratio, unbound DNA could still be visualized on the gel. Complete neutralization was achieved at charge ratios from approximately 2:1 and greater. By 2:1 and 4:1 charge ratios, no unbound DNA can be seen suggesting there is sufficient polymer complexed with DNA to neutralize the charge and prevent migration.

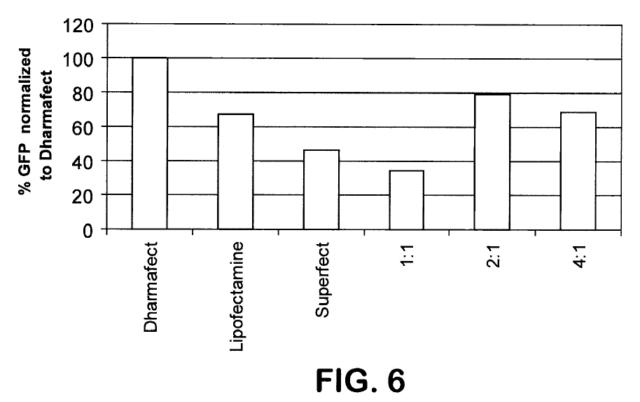

Mouse hepatocyte FL83B cells were seeded in 24-well plates at a density of 30,000 cells/well. The polymer: DNA complexes made in serum free media were added to FL83B cells at the above charge ratios. The complexes were left on the cells for 18-24 hours at 37° C. The cells were then re-fed with fresh media supplemented with 10% fetal bovine serum and incubated for an additional 48 hours at 37° C. Cells positive for green fluorescent protein (Aldevron, Fargo, N. Dak.) expression were observed microscopically and were quantified by flow cytometry on a BD FACSCanto™ (BD Biosciences, Franklin Lakes, N.J.). GFP expression is shown in FIG. 5. Surprisingly, MVPEA-Arg(Ome).HCl had the highest transfection efficiencies of all the polymers tested. However, such efficiency was only 20% of that achieved by the commercial reagent. Reducing the transfection time and including serum in the media improved transfection efficiency as shown in FIG. 6 and the efficiency improved to 80% as compared to the commercial reagent, Dharmafect®.

Comparison of Transfection Efficiency with GFP in Human Cervical Cancer Cells (ATCC) and Human Coronary Artery Endothelial Cells (Cambrex)

Human Coronary Artery Endothelial Cells were purchased from Cambrex BioScience (Walkersville, Md.). DNA was complexed with PEA-Arg(OMe)HCl (formula VI) at charge ratios of 6:1 polymer to DNA as described above. Polymer: DNA complexes were made in 20 mM HEPES buffer pH 7. Transfection capacity was compared with commercial transfection reagents Dharmafect 1 (Dharmacon, Lafayette, Colo.), Lipofectamine (Invitrogen, Carlsbad, Calif.) Superfect (Qiagen, Valencia, Calif.) JetPEI (Polyplus-Transfection, New York, N.Y.), and LT-1 (Mirus, Madison, Wis.).

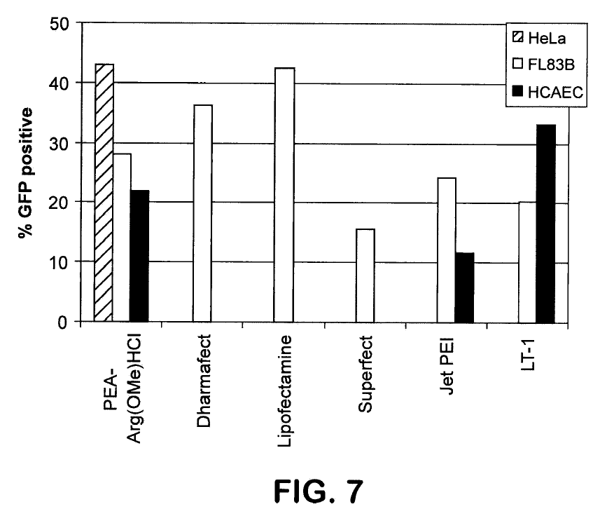

HeLa, Human cervical cancer cells, HCAEC, Human coronary artery endothelial cells and FL83B, Mouse liver cells were seeded in 24-well plates at a density of 10,000, 10,000 and 30,000 cells/well, respectively. The polymer: DNA complexes were added to cells at a concentration of 1 μg DNA/well in media supplemented with 10% fetal bovine serum. The complexes were left on the cells for 72 hours at 37° C. Cells positive for green fluorescent protein (Aldevron, Fargo, N. Dak.) expression were observed microscopically and were quantified by flow cytometry on a BD FACSCanto™. GFP expression is shown in FIG. 7. Compared to commercial reagents, PEA-Arg(OMe)HCl had advantageous transfection efficiencies for HeLa cells, and transfection efficiency was comparable in FL83B cells. HCAEC were only transfected by PEA-Arg(OMe)HCl, JetPEI and LT-1.

### EXAMPLE 3

### siRNA Transfection and Expression

A panel of siRNAs against Sjorgen's syndrome B (SSB) was purchased from Dharmacon and Ambion (Austin, Tex.). The siRNAs were reconstituted in 1×siRNA buffer (6 mM HEPES pH 7.5, 20 mM KCl, 0.2 mM MgCl₂) to 20 μM and stored at −20° C. The panel was screened for down regulation of SSB gene expression and compared to a commercially available transfection reagent, Dharmafect®.

siRNA was formulated with PEA-Arg(OMe) at charge ratios of 1:1, 2:1, and 4:1 polymer to siRNA. Formation of the polymer:siRNA complex was confirmed by running an agarose gel retardation assay to detect formation of polymer:siRNA condensates at four charge ratios as follows: Lane 1=1 kb Plus DNA ladder; Lane 2=0.6 μg siRNA only; Lane 3=PEA only at 6:0 charge ratio; Lane 4=1:1 charge ratio PEA:siRNA; Lane 5=2:1 charge ratio PEA:siRNA; Lane 6=4:1 charge ratio PEA:siRNA; Lane 7=6:1 charge ratio PEA:siRNA. Observation of a photomicrograph of the results of the gel retardation assay of PEA-Arg(OMe)HCl complexed with siRNA at various charge ratios revealed that at a 1:1 charge ratio, unbound siRNA was observed in the agarose gel. However, at charge ratios of 2:1, 4:1, and 6:1, the siRNA was fully complexed with polymer and no migration was observed. Formation of the neutralized polymer:siRNA complex was also confirmed by zeta potential assay and DLS as shown in Table 2 herein.

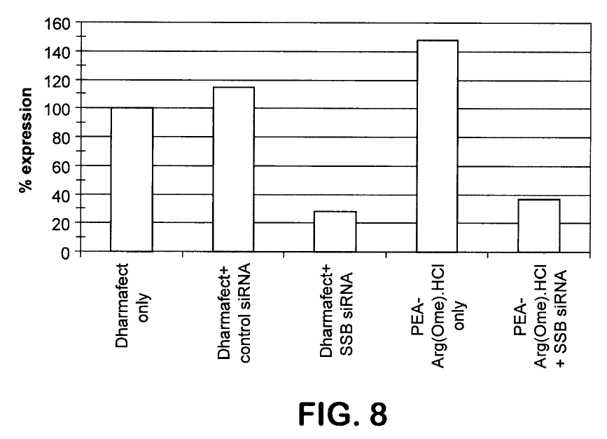

Polymer: siRNA complexes were made in serum free media, allowed to complex for 40 minutes, followed by the addition of fresh media. The complexes were added to FL83B cells and transfected at a final DC03 concentration of 100 nM for 18-24 hours at 37° C. After 24 hours fresh media was added and cells were incubated for an additional 24 hours at 37° C. Cells were harvested and RNA isolated using an RNeasy RNA isolation kit (Qiagen, Valencia, Calif.). Gene expression was measured by quantitative PCR. The results of this experiment, (FIG. 8) showed that transfection of siRNA complexed to PEA-Arg(OMe).HCl or to Dharmafect® resulted in approximately 70% down regulation of SSB expression.

Cytotoxicity of PEA Polymer: siRNA Complex Relative to Commercial Transfection Reagents

Cytotoxicity of the polymer: siRNA complex was measured as described in previous example for polymer: DNA complexes. Briefly, polymer: siRNA complexes were made by adding a volume of 10 mg/mL polymer suspension to a volume of siRNA to yield a final siRNA concentration of 100 nM in 25 mM Hepes pH 7. These complexes were added to cells in a 24 well plate at 37° C. under 5% CO₂. Cytotoxicity of the polymer: siRNA complex was measured at 24 and 48 hours by Vialight™ assay. As shown by the data summarized in FIG. 9, viability of FL83B cells in the presence of invention polymer:siRNA complexes was as advantageous as in the best commercially available transfection reagents.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A gene transfer composition comprising at least one poly nucleic acid in a soluble complex with a cationic biodegradable polymer comprising at least one of the following:
a PEA polymer having a chemical formula described by general structural formula (I), 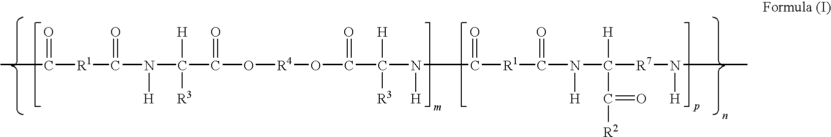
wherein n ranges from about 15 to about 150, m ranges about 0.1 to 0.9; p ranges from about 0.9 to 0.1;
R¹ is independently selected from the group consisting of (C₂-C₂₀) alkylene, (C₂-C₂₀) alkenylene, α,ω-bis(4-carboxyphenoxy)-(C₁-C₈) alkane, and α,ω-alkylene dicarboxylates of structural formula (II) and combinations thereof; and wherein R⁵ in Formula (II) is independently selected from (C₂-C₁₂) alkylene, and (C₂-C₁₂) alkenylene, and R⁶ in Formula (II) is independently selected from the group consisting of (C₂-C₁₂) alkylene, (C₂-C₁₂) alkenylene, and (C₂-C₈) alkyloxy (C₂-C₂₀) alkylene, 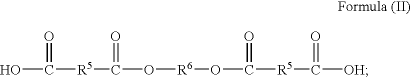
R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and 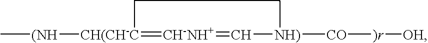
(polyhistidine), wherein r ranges from about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (III), and combinations thereof; and 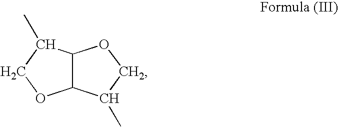
R⁷ is independently (C₂-C₂₀) alkyl or (C₂-C₂₀) alkenyl;
or a PEUR polymer having a chemical structure described by general structural formula (IV) 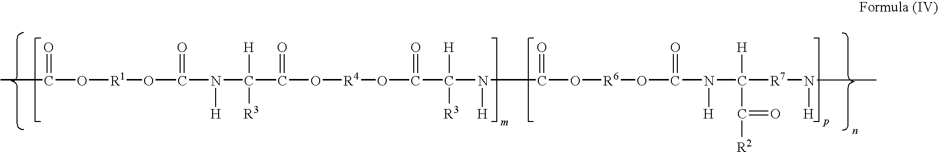
wherein n ranges from about 15 to about 150, m ranges from about 0.1 to 0.9; p ranges from about 0.9 to 0.1;
R¹ is independently selected from the group consisting of (C₂-C₂₀) alkylene, (C₂-C₂₀) alkenylene, α,ω-bis(4-carboxyphenoxy)-(C₁-C₈) alkane, and α,ω-alkylene dicarboxylates of structural formula (II) and combinations thereof; and wherein R⁵ in Formula (II) is independently selected from (C₂-C₁₂) alkylene, and (C₂-C₁₂) alkenylene, and R⁶ in Formula (II) is independently selected from the group consisting of (C₂-C₁₂) alkylene, (C₂-C₁₂) alkenylene, and (C₂-C₈) alkyloxy (C₂-C₂₀) alkylene,
R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹-(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and 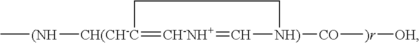
(polyhistidine), wherein r ranges from about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ and R⁶ are each independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof; and
R⁷ is independently (C₂-C₂₀) alkyl or (C₂-C₂₀) alkenyl;
or a PEU polymer having a chemical formula described by general structural formula (V): 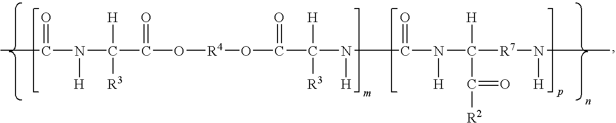
wherein n ranges from about 15 to about 150, m ranges about 0.1 to 0.9; p ranges from about 0.9 to 0.1;
R² is independently selected from the group consisting of hydroxyl, —O—(C₁-C₁₂) oxyalkyl, —O—(C₁-C₁₂) oxyalkyl (C₆-C₁₀) aryl and a protecting group, except that sufficient of the R² to neutralize charge on the poly nucleic acid is selected from the group of cationic residues consisting of —R⁸—R⁹—NH—C(═NH₂⁺)NH₂, —R⁸—R⁹—NH₂⁺, —R⁸—R⁹—(4-methylene imidazolinium), —(NH—CH(CH₂CH₂CH₂—NHC(═NH₂⁺)NH₂)CO-)r-OH, (polyarginine), —(NH—CH(CH₂CH₂CH₂CH₂NH₃⁺)—CO-)r-OH, (polylysine), —(NH—CH(CH₂CH₂CH₂NH₃⁺)—CO-)r-OH, (polyornithine) and 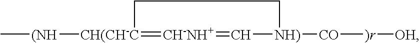
(polyhistidine), wherein r ranges form about 2 to about 50; R⁸ is —O—, —S— or —NR¹⁰—, wherein R¹⁰ is selected from the group consisting of hydrogen, (C₁-C₈) alkyl, —CH(CO(C₁-C₈) alkyloxy)-, —CH(CO(PG))-; R⁹ is (C₁-C₁₂) alkylene or (C₃-C₁₂) alkenylene, and PG is a protecting group;
R³ is independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II); and
R⁷ is independently (C₂-C₂₀) alkyl or (C₂-C₂₀) alkenyl.

2. The composition of claim 1, wherein at least one of the cationic residues is —NHCH(COOMe)-(CH₂)₃NHC(═NH₂⁺)NH₂

3. The composition of claim 1, wherein at least one of the cationic residues is —NH—(CH₂)₄NHC(═NH₂⁺)NH₂.

4. The composition of claim 1, wherein the PEA polymer is described by the following general structural formula:

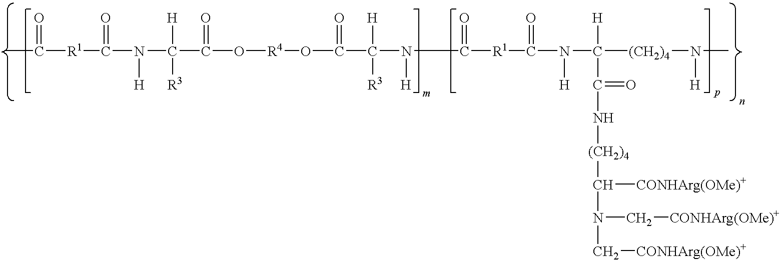

5. The composition of claim 1, wherein the PEA polymer is described by the following general structural formula:

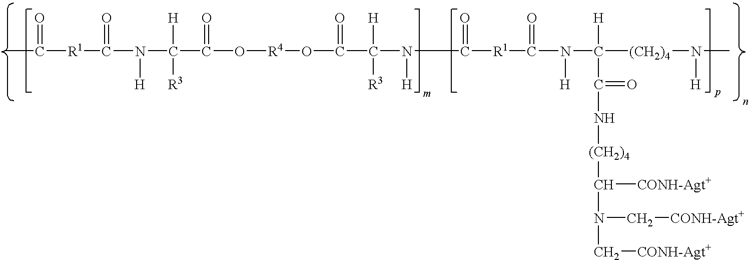

6. The composition of claim 1, wherein at least one of the cationic residues is —(NH—CH(CH₂CH₂CH₂—NHC(═NH₂⁺)NH₂)CO-)r, wherein r ranges from about 2 to about 25

7. The composition of claim 1, wherein the R⁷s comprise —(CH₂)₄—.

8. The composition of claim 1, wherein at least one of the cationic residues is 4-methylene imidazolinium ion as a residue of histidine methyl ester:

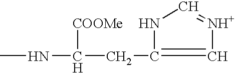

9. The composition of claim 1, further comprising at least one acidic counter-ion associated with the polymer.

10. The composition of claim 9, wherein the associated acidic counter ion has a pKa from about −7 to +5.

11. The composition of claim 1, wherein the poly nucleic acid comprises a gene encoding a therapeutic polypeptide.

12. The composition of claim 11, wherein the poly nucleic acid further comprises plasmid DNA suitable for expressing the gene.

13. The composition of claim 12, wherein the plasmid DNA is suitable for expression of the gene in a mammalian target cell.

14. The composition of claim 1, wherein charge ratio of the polymer to the poly nucleic acid is from about 2:1 to about 4:1.

15. The composition of claim 1, wherein the poly nucleic acid comprises RNA.

16. The composition of claim 15, wherein the RNA comprises antisense poly nucleic acid that is complimentary to an mRNA that encodes a target protein.

17. The composition of claim 1, wherein the poly nucleic acid comprises iRNA for suppression of a target gene in a target cell.

18. The composition of claim 17, wherein the iRNA forms siRNA.

19. The composition of claim 1, wherein the DNA is cDNA encoding a therapeutic polypeptide.

20. The composition of claim 19 wherein there is a polymer:poly nucleic acid weight ratio of about 1:1 to about 2000.1.

21. A method for transfecting a target cell comprising:
incubating a target cell with a composition of claim 1 in solution under conditions and for a time suitable to cause the composition to enter the target cell so as to transfect the target cell with the poly nucleic acid in the composition.

22. The method of claim 21, wherein at least one of the cationic residues is —NHCH(COOMe)-(CH₂)₃NHC(═NH₂⁺)NH₂.

23. The method of claim 21, wherein at least one of the cationic residues is —NH—(CH₂)₄NHC(═NH₂⁺)NH₂.

24. The method of claim 21, wherein the R²s comprise:

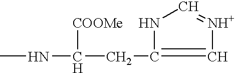

25. The method of claim 21, wherein the poly nucleic acid comprises a gene encoding a therapeutic polypeptide.

26. The method of claim 25, wherein the poly nucleic acid further comprises plasmid DNA suitable for expressing the gene.

27. The method of claim 26, wherein the plasmid DNA is suitable for expression of the gene in a mammalian target cell.
